(19) United States
(12) Patent Application Publication
(10) Pub. No.: US 2023/0079748 A1
(43) Pub. Date: Mar. 16, 2023

(54) PREPARATION METHOD, PRODUCT, AND APPLICATION OF CIRCULATING TUMOR DNA REFERENCE SAMPLES

(71) Applicant: SeekIn, Inc., Shenzhen (CN)

(72) Inventors: Guolin Zhong, Shenzhen (CN); Mao Mao, Shenzhen (CN); Shiyong Li, Shenzhen (CN); Dandan Zhu, Zhengzhou (CN); Yan Chen, Shenzhen (CN); Yumin Feng, Shenzhen (CN); Wei Wu, Shenzhen (CN)

(21) Appl. No.: 17/901,431

(22) Filed: Sep. 1, 2022

(30) Foreign Application Priority Data
Sep. 1, 2021 (CN) 202111029341.8

(51) Int. Cl. C12Q 1/6886

(52) U.S. Cl. CPC C12Q 1/6886; C12Q 1/6806

(57) ABSTRACT

The present disclosure relates to methods of preparing circulating tumor DNA (ctDNA) reference samples including: inducing apoptosis in tumor cells to obtain DNA fragments and then extracting DNA from the tumor cells to obtain the circulating tumor DNA reference samples. The methods to prepare ctDNA reference samples disclosed herein are simple and easy to use, suitable for various tumor cells, and the variant information can be retained to simulate the ctDNA in animals. In some embodiments, the reference samples can facilitate assay calibration and evaluation.

### CLAIM OF PRIORITY

This application claims the benefit of Chinese Patent Application App. No. 202111029341.8, filed on Sep. 1, 2021. The entire contents of the foregoing application are incorporated herein by reference.

### TECHNICAL FIELD

The present disclosure belongs to the biotechnology field, and in particular, the present disclosure relates to methods of preparing circulating tumor DNA as reference samples, methods of production and application.

### BACKGROUND

Circulating cell-free DNA (cfDNA) is continuously released into blood following apoptosis or necrosis, and is usually about 150 to 200 base pairs with a half-life of about 0.5-1 hours. In healthy subjects, cfDNA concentration is minimal, usually 10-15 ng per 1 mL of plasma, whereas cfDNA concentration is significantly increased in cancer patients. The DNA released from tumor cells measured in peripheral blood is also known as circulating tumor DNA (ctDNA), and ctDNA is a subgroup of cfDNA. The proportion of ctDNA in cfDNA varies greatly among cancer patients, from as little as 0.1% to more than 90%. With the emergence of the Next-Generation Sequencing (NGS) technology, massively parallel sequencing has become possible. Compared with the Sanger sequencing technology, NGS technology has many advantages, e.g., fast speed, high accuracy, low cost and wide coverage. With the continuous maturation of NGS technology, the technology of cfDNA detection using NGS has been widely used in cancer early screening and diagnosis, medication guidance, prognostic stratification, recurrence monitoring and treatment response monitoring. Previous studies on cfDNA have focused on the analysis of different types of mutations (point mutations, insertions, deletions, etc.), copy number alterations as well as genetic correlations and polymorphisms. A growing number of studies now show that the cfDNA fragmentation pattern differs significantly between normal individuals and cancer patients, and that the pattern differs among different tissue of origins. By taking cfDNA fragmentation patterns into account, it is possible to assess whether the subject has malignant lesion and to trace its tissue of origin.

However, reference samples are required for validating the performance of detection somatic mutations, including single nucleotide variant (SNV), insertions and deletions (indels), structural variation (SV), copy number variation (CNV) and fragment size of cfDNA. The reference sample serves as a “ruler” throughout the process of method development, optimization, and performance confirmation, which reflecting the true performance of the method before it is applied in clinical. Therefore, well-characterized reference samples can be valuable in assay development, test validation, internal quality control, and external proficiency tests.

The ideal reference samples are derived from clinical samples, but clinical samples are valuable research materials limited in availability, making it difficult to serve as a long-term source of reference samples.

Thus, it is crucial to develop circulating tumor DNA reference samples to replace clinical samples. The reference samples should have high consistency with clinical samples and are simple to prepare.

### SUMMARY

Recent advances in technologies, e.g., next-generation sequencing (NGS), have enabled the detection of genetic signatures (e.g., point mutation, copy number variation, structural variation and fragmentation pattern) of cancer present at low levels in ctDNA in blood. Growing numbers of laboratory-developed liquid biopsy tests based on such technologies have become commercially available for clinical usage. However, the accuracy, reliability, and preciseness are critical for evaluating the performance of NGS in measuring low levels of mutations in ctDNA. Therefore, well-characterized quality control samples of known variations at known concentrations can be valuable.

The development of reference samples for cfDNA is complicated by the degraded nature of the DNA strands in blood. The biological origin of cfDNA has not been fully investigated, but the cfDNA size distribution suggests that the DNA molecules are protected by the binding of proteins (in form of nucleosomes) from digestion by nucleases in the cell or blood, producing a degradation pattern similar to the DNA degradation that occurs during apoptosis. In this disclosure, the inventors generated synthetic cfDNA as quality control materials by extracting DNA from apoptosis cells. The reference samples described herein can simulate the real plasma cfDNA fragmentation pattern to the maximum extent and do not affect the detection of point mutation, copy number variation and structural variation. The reference samples described herein are valuable in assay development, test validation, internal quality control, and efficacy tests.

In one aspect, the disclosure is related to a method of preparing a circulating tumor DNA reference sample, the method comprising: (1) inducing apoptosis in tumor cells; and (2) extracting DNA from the tumor cells to obtain the circulating tumor DNA reference sample. In some embodiments, the tumor cells are incubated with an apoptosis inducer (e.g., in the culture medium) to induce apoptosis. In some embodiments, the apoptosis inducer can bind to the topoisomerase-DNA complex during DNA replication to prevent DNA strand reassembly and/or cause DNA double strand break. In some embodiments, the tumor cells are incubated with the apoptosis inducer in the culture medium for 2-8 hours. In some embodiments, the tumor cells are incubated with the apoptosis inducer in the culture medium for about 5 hours. In some embodiments, the apoptosis inducer is selected from the group consisting of camptothecin (CPT), As203, notopterol and gracillin. In some embodiments, the apoptosis inducer is CPT. In some embodiments, the concentration of the apoptosis inducer is about 5-15 μM. In some embodiments, the concentration of the apoptosis inducer is about 10 μM.

In one aspect, the disclosure is related to a method of preparing a circulating tumor DNA reference sample, the method comprising: (1) inducing apoptosis in tumor cells by treating tumor cells with CPT at a concentration of about 10 μM for 2-8 hours (e.g., about 5 hours); and (2) extracting DNA from the tumor cells to obtain the circulating tumor DNA reference sample.

In one aspect, the disclosure is related to a circulating tumor DNA reference sample obtained using the method described herein.

In one aspect, the disclosure is related to a method for determining the quality of the circulating tumor DNA reference sample described herein, the method comprising: (1) providing a first DNA library of DNA extracted from tumor cells that are not treated with an apoptosis inducer; (2) providing a second DNA library by sequencing the circulating tumor DNA reference sample; (3) identifying one or more genetic variations in the first DNA library and one or more genetic variations in the second DNA library; and (4) comparing the one or more genetic variations in the first and second DNA libraries. In some embodiments, consistency of the genetic variations in the first and second DNA libraries indicates a good quality of the circulating tumor DNA reference sample. In some embodiments, the one or more genetic variations are selected from the group consisting of single nucleotide variations (e.g., point mutations), structural variations, copy number variations, and/or fragmentation pattern variations. In some embodiments, the methods described herein further comprises, prior to step (1): determining and comparing the size distribution of the circulating tumor DNA reference sample and the size distribution of the cell-free DNA (cfDNA) from plasma of a subject. In some embodiments, the size distributions of the circulating tumor DNA reference sample and the cfDNA share a fragmentation pattern having one or more of the following features: (1) the fragmentation pattern comprises a main peak representing nucleosome monomers with a length of about 166 bp; (2) the fragmentation pattern comprises one or more sub-peaks representing complexes of nucleosome monomers (e.g., dimers and trimers); and (3) the fragmentation pattern comprises one or more minor sub-peaks with a length of less than 150 bp.

In one aspect, the disclosure is related to a method of predicting cancer using the circulating tumor DNA reference sample as described herein, or the circulating tumor DNA reference sample prepared by the method described herein.

In one aspect, the disclosure is related to a method of predicting cancer, comprising: (1) determining the size distribution of the cell-free DNA (cfDNA) from plasma of a subject; (2) determining the size distribution of the circulating tumor DNA reference sample as described herein; and (3) comparing the size distribution of the cfDNA in (1) and the size distribution of the circulating tumor DNA reference sample in (2). In some embodiments, a matching fragmentation pattern of the size distribution of the cfDNA in step (1) and the size distribution of the circulating tumor DNA reference sample in step (2) indicates existence of cancer in the subject, In some embodiments, the fragmentation pattern is matched when the Pearson correlation coefficient is at least 0.5 for fragments between 50-166 bp between the size distribution of the cfDNA in step (1) and the size distribution of the circulating tumor DNA reference sample in step (2). In some embodiments, the subject is a human patient diagnosed with cancer, suspected to have cancer, or having a risk to have cancer.

In one aspect, the disclosure is related to a method of validating an assay, comprising: (1) providing a first DNA library of DNA prepared from a first type of cells (e.g., tumor cells) treated with an apoptosis inducer, in some embodiments, the first type of cells have one or more mutations at a chromosomal site; (2) providing a second DNA library of DNA prepared from a second type of cells (e.g., normal cells) treated with the apoptosis inducer, in some embodiments, the second type of cells have no mutation at the chromosomal site; (3) constructing a third DNA library of DNA prepared from a test sample; and (4) detecting the one or more mutations from the constructed DNA libraries. In some embodiments, detection of the one or more mutations from the first DNA library and no detection of the one or more mutations from the second DNA library indicate the assay is validated. In some embodiments, no detection of the one or more mutations from the first DNA library or detection of the one or more mutations from the second DNA library indicates the assay is not validated.

In one aspect, the disclosure is related to a method of determining the limit of detection (LOD) of mutation frequency of an assay, comprising: (1) providing DNA extracted from a first type of cells treated with an apoptosis inducer, in some embodiments, the first type of tumor cells have one or more mutations at a chromosomal site; (2) providing DNA extracted from a second type of cells treated with the apoptosis inducer, in some embodiments, the second type of tumor cells have no mutation at the chromosomal site; (3) mixing the DNA from step (1) and step (2) at different ratios to obtain a series of DNA samples; (4) constructing one or more DNA libraries from the series of DNA samples; and (5) determining the frequency of the one or more mutations from the constructed DNA libraries. In some embodiments, the LOD of mutation frequency of the assay can be determined by the frequency of the one or more mutations from the constructed DNA libraries. In some embodiments, the series of DNA samples have different mutation frequencies. In some embodiments, the first type of cells are tumor cells and/or the second type of cells are normal cells. In some embodiments, the test sample is from a human patient diagnosed with cancer, suspected to have cancer, or having a risk to have cancer. In some embodiments, the test sample is plasma. In some embodiments, the test sample contains circulating cell-free DNA (cfDNA), e.g., circulating tumor DNA (ctDNA).

In one aspect, the disclosure is related to a method of validating an assay, comprising: (1) providing a first DNA library of DNA extracted from tumor cells treated with an apoptosis inducer, in some embodiments, the tumor cells have one or more mutations at a chromosomal site; (2) constructing a second DNA library of DNA prepared from a test sample; and (3) detecting the one or more mutations from the constructed DNA libraries. In some embodiments, detection of the one or more mutations from the first DNA library indicates the assay is validated. In some embodiments, no detection of the one or more mutations from the first DNA library indicates the assay is not validated. In some embodiments, the method described herein further comprises: providing a third DNA library of DNA extracted from normal cells treated with the apoptosis inducer, in some embodiments, the normal cells have no mutation at the chromosomal site. In some embodiments, no detection of the one or more mutations from the third DNA library indicates the assay is validated. In some embodiments, detection of the one or more mutations from the third DNA library indicates the assay is not validated.

In one aspect, the disclosure is related to a method for mimicking human plasma with different DNA mutation frequency, comprising: adding the circulating tumor DNA reference sample as described herein, or the circulating tumor DNA reference sample prepared by the method as described herein into artificial plasma.

In one aspect, the disclosure is related to a cancer prediction kit, comprising the circulating tumor DNA reference sample as described herein, or the circulating tumor DNA reference sample prepared by the method as described herein.

As disclosed herein, the term “CPT” refers to camptothecin, a cancer treatment drug.

As disclosed herein, the term single-nucleotide variant (SNV), also known as single-nucleotide polymorphism (SNP), is the variant of a single nucleotide that occurs at a specific genomic position.

As disclosed herein, “structural variation (SV)” is generally defined as a region of DNA approximately 1 kb and larger in size and can include inversions, balanced translocations or genomic imbalances (insertions and deletions), commonly referred to as copy number variations (CNVs).

As disclosed herein, the term “copy number variation (CNV)” is an important molecular mechanism for many human diseases (e.g., cancer, genetic diseases, cardiovascular diseases). It usually refers to the genomic structural variation of DNA fragments of 1 kb or larger in length, including microscopic and submicroscopic DNA deletions, insertions, and duplications.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be predicted using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the tumor or cancer described herein is lymphoma, non-small cell lung cancer, cervical cancer, leukemia, ovarian cancer, nasopharyngeal cancer, breast cancer, endometrial cancer, colon cancer, rectal cancer, gastric cancer, bladder cancer, glioma, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myeloproliferation abnormal syndromes, and sarcomas. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T-cell lymphoma, and Waldenstrom macroglobulinemia. In some embodiments, the sarcoma is selected from the group consisting of osteosarcoma, Ewing sarcoma, leiomyosarcoma, synovial sarcoma, soft tissue sarcoma, angiosarcoma, liposarcoma, fibrosarcoma, rhabdomyosarcoma , and chondrosarcoma.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A-1C show the results of different DNA fragment size comparisons. FIG. 1A shows DNA fragmentation by ultrasonication. FIG. 1B shows DNA obtained from artificially-induced apoptotic cells. FIG. 1C shows plasma cfDNA from a cancer patient. The dashed lines stand for fragments of about 80 bp, 91 bp, 102 bp, 111 bp, 122 bp, and 134 bp.

FIGS. 2A-2G show fragmented DNA derived from different cells using different apoptosis-inducing methods. FIGS. 2A-2D show the distribution of DNA fragments obtained from HL-60 resistance cells after CPT treatment for 5 hours, ATRA treatment for 3 days, high-density culture for 3 days, and CPT treatment for 24 hours, respectively. FIGS. 2E-2G show the distribution of DNA fragments obtained from NB-4 cells after CPT treatment for 5 hours, ATRA treatment for 3 days, and high-density culture for 3 days, respectively. The dashed lines stand for fragments of about 80 bp, 91 bp, 102 bp, 111 bp, 122 bp, and 134 bp.

FIG. 3 shows the consistency analysis of copy number variation between the cfDNA reference samples and paired NB4 cell line genomic DNA; x-axis show the information of copy number variation from cfDNA reference samples, y-axis show the information of copy number variation from paired NB4 cell line genomic DNA.

### DETAILED DESCRIPTION

It was previously found that the fragmentation characteristics of cell-free DNA from plasma can be completely different from that of human genomic DNA. Specifically, cfDNA reference samples are usually prepared by sonication or enzymatic fragmentation to shear human genomic DNA into 100-200 bp fragments. But the sequence information, fragment size and random distribution characteristics of the DNA fragments yielded by these methods are different from those obtained from the plasma cfDNA. The most commonly used enzyme digestion tool at present is micrococcal nuclease (MNase), which degrades DNA in the nucleosome junction region and releases individual nucleosomes. The DNA fragments obtained by this method are shorter than the cfDNA, where the main peak of length distribution of the former is about 146 bp while that of the latter is about 166 bp. Moreover, the MNase enzymatic digestion method has some limitations. Firstly, MNase has a digestion bias for A-T-rich regions, resulting in a decreased presentation of nucleosomes in the A-T-rich regions; secondly, MNase cannot break precisely at the nucleosome boundary, which leads to differences in determining the open position of chromatin from the real situation; thirdly, MNase is biased to digest fragile nucleosomes. Therefore, MNase is not a good digestion tool to prepare cfDNA reference samples. However, it is particularly important to prepare a reference sample that can be used as a quality control for cfDNA variant information detection.

### Sample Preparation

Provided herein are methods and compositions for analyzing nucleic acids. In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, cell or tissue origins, tumor origins, cancer origins, sample origins, subject origins, fetal origins, maternal origins), or combinations thereof.

Nucleic acid or a nucleic acid mixture described herein can be isolated from a sample obtained from a subject. A subject can be any living or non-living organism, including but not limited to a human, a non-human animal, a mammal, a plant, a bacterium, a fungus or a virus. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject can be a male or female.

Nucleic acid can be isolated from any type of suitable biological specimen or sample (e.g., a test sample). A sample or test sample can be any specimen that is isolated or obtained from a subject (e.g., a human subject). Non-limiting examples of specimens include fluid or tissue from a subject, including, without limitation, blood, serum, umbilical cord blood, chorionic amniotic fluid, cerebrospinal fluid, spinal fluid, lavage fluid (e.g., bronchoalveolar, gastric, peritoneal, ductal, ear, arthroscopic), biopsy sample, celocentesis sample, fetal cellular remnants, urine, feces, sputum, saliva, nasal mucous, prostate fluid, lavage, semen, lymphatic fluid, bile, tears, sweat, breast milk, breast fluid, embryonic cells and fetal cells (e.g. placental cells).

In some embodiments, a biological sample can be blood, plasma or serum. As used herein, the term “blood” encompasses whole blood or any fractions of blood, such as serum and plasma. Blood or fractions thereof can comprise cell-free or intracellular nucleic acids. Blood can comprise buffy coats. Buffy coats are sometimes isolated by utilizing a ficoll gradient. Buffy coats can comprise white blood cells (e.g., leukocytes, T-cells, B-cells, platelets). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation. A fluid or tissue sample from which nucleic acid is extracted can be acellular (e.g., cell-free). In some embodiments, a fluid or tissue sample can contain cellular elements or cellular remnants. In some embodiments, cancer cells or tumor cells can be included in the sample.

A sample often is heterogeneous. In many cases, more than one type of nucleic acid species is present in the sample. For example, heterogeneous nucleic acid can include, but is not limited to, cancer and non-cancer nucleic acid, pathogen and host nucleic acid, and/or mutated and wild-type nucleic acid. A sample may be heterogeneous because more than one cell type is present, such as a cancer and non-cancer cell, or a pathogenic and host cell.

In some embodiments, the sample comprise cell free DNA (cfDNA) or circulating tumor DNA (ctDNA). As used herein, the term “cell-free DNA” or “cfDNA” refers to DNA that is freely circulating in the bloodstream. These cfDNA can be isolated from a source having substantially no cells. In some embodiments, these extracellular nucleic acids can be present in and obtained from blood. Extracellular nucleic acid often includes no detectable cells and may contain cellular elements or cellular remnants. Non-limiting examples of acellular sources for extracellular nucleic acid are blood, blood plasma, blood serum and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species. For example, blood serum or plasma from a person having cancer can include nucleic acid from cancer cells and nucleic acid from non-cancer cells. As used herein, the term “circulating tumor DNA” or “ctDNA” refers to tumor-derived fragmented DNA in the bloodstream that is not associated with cells. ctDNA usually originates directly from the tumor or from circulating tumor cells (CTCs). The circulating tumor cells are viable, intact tumor cells that shed from primary tumors and enter the bloodstream or lymphatic system. The ctDNA can be released from tumor cells by apoptosis and necrosis (e.g., from dying cells), or active release from viable tumor cells (e.g., secretion). Studies show that the size of fragmented ctDNA is predominantly 166 bp long, which corresponds to the length of DNA wrapped around a nucleosome plus a linker. Fragmentation of this length might be indicative of apoptotic DNA fragmentation, suggesting that apoptosis may be the primary method of ctDNA release. Thus, in some embodiments, the length of ctDNA or cfDNA can be at least or about 70, 80, 90, 100, 110, 120, 130, 140, 150, 160, 170, 180, 190, or 200 bp. In some embodiments, the length of ctDNA or cfDNA can be less than about 70, 80, 90, 100, 110, 120, 130, 140, 150, 160, 170, 180, 190, or 200 bp. In some embodiments, the cell-free nucleic acid is of a length of about 500, 250, or 200 base pairs or less.

The present disclosure provides methods of separating, enriching and analyzing cell free DNA or circulating tumor DNA found in blood as a non-invasive means to detect the presence and/or to monitor the progress of a cancer. Thus, the first steps of practicing the methods described herein are to obtain a blood sample from a subject and extract DNA from the subject.

A blood sample can be obtained from a subject (e.g., a subject who is suspected to have cancer). The procedure can be performed in hospitals or clinics. An appropriate amount of peripheral blood, e.g., typically between 1 and 50 ml (e.g., between 1 and 10 ml), can be collected. Blood samples can be collected, stored or transported in a manner known to the person of ordinary skill in the art to minimize degradation or the quality of nucleic acid present in the sample. In some embodiments, the blood can be placed in a tube containing EDTA to prevent blood clotting, and plasma can then be obtained from whole blood through centrifugation. Serum can be obtained with or without centrifugation-following blood clotting. If centrifugation is used then it is typically, though not exclusively, conducted at an appropriate speed, e.g., 1,500-3,000×g. Plasma or serum can be subjected to additional centrifugation steps before being transferred to a fresh tube for DNA extraction.

In addition to the acellular portion of the whole blood, DNA can also be recovered from the cellular fraction, enriched in the buffy coat portion, which can be obtained following centrifugation of a whole blood sample.

There are numerous known methods for extracting DNA from a biological sample including blood. The general methods of DNA preparation (e.g., described by Sambrook and Russell, Molecular Cloning: A Laboratory Manual 3d ed., 2001) can be followed; various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), may also be used to obtain DNA from a blood sample.

cfDNA purification is prone to contamination due to ruptured blood cells during the purification process. Because of this, different purification methods can lead to significantly different cfDNA extraction yields. In some embodiments, purification methods involve collection of blood via venipuncture, centrifugation to pellet the cells, and extraction of cfDNA from the plasma. In some embodiments, after extraction, cell-free DNA can be about or at least 50% of the overall nucleic acid (e.g., about or at least 50%, 60%, 70%, 80%, 90%, 91%, 92%, 93%, 94%, 95%, 96%, 97%, 98%, or 99% of the total nucleic acid is cell-free DNA).

The nucleic acid that can be analyzed by the methods described herein include, but are not limited to, DNA (e.g., complementary DNA (cDNA), genomic DNA (gDNA), cfDNA, or ctDNA), ribonucleic acid (RNA) (e.g., message RNA (mRNA), short inhibitory RNA (siRNA), ribosomal RNA (rRNA), transfer RNA (tRNA), or microRNA), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, or double-stranded). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures.

Nucleic acid provided for processing described herein can contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples). In some embodiments, the nucleic acids are from reference samples. In some embodiments, the nucleic acids are from test samples. In some embodiments, the nucleic acids are from tumor cells. In some embodiments, the nucleic acids are from normal cells.

In some embodiments, the nucleic acid can be extracted, isolated, purified, partially purified or amplified from the samples before sequencing. In some embodiments, nucleic acid can be processed by subjecting nucleic acid to a method that generates nucleic acid fragments. Fragments can be generated by a suitable method known in the art, and the average, mean or nominal length of nucleic acid fragments can be controlled by selecting an appropriate fragment-generating procedure. In certain embodiments, nucleic acid of a relatively shorter length can be utilized to analyze sequences that contain little sequence variation and/or contain relatively large amounts of known nucleotide sequence information. In some embodiments, nucleic acid of a relatively longer length can be utilized to analyze sequences that contain greater sequence variation and/or contain relatively small amounts of nucleotide sequence information.

### Sequencing and Library Construction

Nucleic acids (e.g., nucleic acid fragments, sample nucleic acid, cell-free nucleic acid, circulating tumor nucleic acids) are sequenced before the analysis.

As used herein, “reads” or “sequence reads” are short nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads).

Sequence reads obtained from cell-free DNA can be reads from nucleic acids derived from normal cells and/or tumor cells. In some embodiments, the nucleic acids include nucleic acids from reference samples and/or test samples as described herein. In some embodiments, the nucleic acids are labeled (e.g., to identify the source of the cells). A mixture of relatively short reads can be transformed by processes described herein into a representation of a genomic nucleic acid present in a subject. In certain embodiments, “obtaining” nucleic acid sequence reads of a sample can involve directly sequencing nucleic acid to obtain the sequence information.

Sequence reads can be mapped and the number of reads or sequence tags mapping to a specified nucleic acid region (e.g., a chromosome, a bin, a genomic section) are referred to as counts. In some embodiments, counts can be manipulated or transformed (e.g., normalized, combined, added, filtered, selected, averaged, derived as a mean, the like, or a combination thereof).

In some embodiments, a group of nucleic acid samples from one individual are sequenced. In certain embodiments, nucleic acid samples from two or more samples, wherein each sample is from one individual or two or more individuals, are pooled and the pool is sequenced together. In some embodiments, a nucleic acid sample from each biological sample often is identified by one or more unique identification tags.

The nucleic acids can also be sequenced with redundancy. A given region of the genome or a region of the cell-free DNA can be covered by two or more reads or overlapping reads (e.g., “fold” coverage greater than 1). Coverage (or depth) in DNA sequencing refers to the number of unique reads that include a given nucleotide in the reconstructed sequence. In some embodiments, a fraction of the genome is sequenced, which sometimes is expressed in the amount of the genome covered by the determined nucleotide sequences (e.g., “fold” coverage less than 1). Thus, in some embodiments, the fold is calculated based on the entire genome. In some embodiments, cell free DNAs are sequenced and the fold is calculated based on the entire genome. Thus, it is easier to compare the amount of sequencing and the amount of sequencing reads that are generated for different projects.

The fold can also be calculated based on the length of the reconstructed sequence (e.g., cfDNA). When the cell free DNA is sequenced with about 1-fold coverage that is calculated based on the reconstructed sequence (e.g., panel sequencing), the number of nucleotides in all unique reads would be roughly the same as the entire nucleotide sequence of the cfDNA in the sample.

In some embodiments, the nucleic acid is sequenced with about 0.1-fold to about 100-fold coverage, about 0.2-fold to 20-fold coverage, or about 0.2-fold to about 1-fold coverage. In some embodiments, sequencing is performed by about or at least 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 200, 300, 400, 500, or 1000 fold coverage. In some embodiments, sequencing is performed by no more than 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 200, 300, 400, 500, or 1000 coverage. In some embodiments, sequencing is performed by no more than 15, 20, 30, 40, 50, 60, 70, 80, 90 or 100 fold coverage.

In some embodiments, the sequence coverage is performed by about or at least 0.2, 0.3, 0.4, 0.5, 0.6, 0.7, 0.8, 0.9, 1, 2, 3, 4, or 5 fold (e.g., as determined by the entire genome). In some embodiments, the sequence coverage is performed by no more than 0.2, 0.3, 0.4, 0.5, 0.6, 0.7, 0.8, 0.9, 1, 2, 3, 4, or 5 fold (e.g., as determined by the entire genome).

In some embodiments, the sequence coverage is performed by about or at least 100, 150, 200, 250, 300, 350, 400, 450, or 500 fold (e.g., as determined by reconstructed sequence). In some embodiments, the sequence coverage is performed by no more than 100, 150, 200, 250, 300, 350, 400, 450, or 500 fold (e.g., as determined by reconstructed sequence).

In some embodiments, a sequencing library can be prepared prior to or during a sequencing process. Methods for preparing the sequencing library are known in the art and commercially available platforms may be used for certain applications. Certain commercially available library platforms may be compatible with sequencing processes described herein. For example, one or more commercially available library platforms may be compatible with a sequencing by synthesis process. In certain embodiments, a ligation-based library preparation method is used (e.g., ILLUMINA TRUSEQ, Illumina, San Diego Calif.). Ligation-based library preparation methods typically use a methylated adaptor design which can incorporate an index sequence at the initial ligation step and often can be used to prepare samples for single-read sequencing, paired-end sequencing and multiplexed sequencing. In certain embodiments, a transposon-based library preparation method is used (e.g., EPICENTRE NEXTERA, Epicentre, Madison Wis.). Transposon-based methods typically use in vitro transposition to simultaneously fragment and tag DNA in a single-tube reaction (often allowing incorporation of platform-specific tags and optional barcodes), and prepare sequencer-ready libraries.

Any sequencing method suitable for conducting methods described herein can be used. In some embodiments, a high-throughput sequencing method is used. High-throughput sequencing methods generally involve clonally amplified DNA templates or single DNA molecules that are sequenced in a massively parallel fashion within a flow cell. Such sequencing methods also can provide digital quantitative information, where each sequence read is a countable “sequence tag” or “count” representing an individual clonal DNA template, a single DNA molecule, bin or chromosome.

Next generation sequencing techniques capable of sequencing DNA in a massively parallel fashion are collectively referred to herein as “massively parallel sequencing” (MPS). High-throughput sequencing technologies include, for example, sequencing-by-synthesis with reversible dye terminators, sequencing by oligonucleotide probe ligation, pyrosequencing and real time sequencing. Non-limiting examples of MPS include Massively Parallel Signature Sequencing (MPSS), Polony sequencing, Pyrosequencing, Illumina (Solexa) sequencing, SOLiD sequencing, Ion semiconductor sequencing, DNA nanoball sequencing, Helioscope single molecule sequencing, single molecule real time (SMRT) sequencing, nanopore sequencing, ION Torrent and RNA polymerase (RNAP) sequencing. Some of these sequencing methods are described e.g., in US20130288244A1, which is incorporated herein by reference in its entirety.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific

Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

The length of the sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median or average length of about 15 bp to 900 bp long (e.g., about or at least 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 110 bp, 120 bp, 130, 140 bp, 150 bp, 200 bp, 250 bp, 300 bp, 350 bp, 400 bp, 450 bp, or 500 bp). In some embodiments, the sequence reads are of a mean, median or average length of about 1000 bp or more. In some embodiments, the sequence reads are of less than 60 bp, 65 bp, 70 bp, 75 bp, 80 bp, 85 bp, 90 bp, 95 bp, 100 bp, 110 bp, 120 bp, 130, 140 bp, 150 bp, 200 bp, 250 bp, 300 bp, 350 bp, 400 bp, 450 bp, or 500 bp are removed because of poor quality.

Mapping nucleotide sequence reads (i.e., sequence information from a fragment whose physical genomic position is unknown) can be performed in a number of ways, and often comprises alignment of the obtained sequence reads with a matching sequence in a reference genome (e.g., Li et al., “Mapping short DNA sequencing reads and calling variants using mapping quality score,” Genome Res., 2008 Aug. 19.) In such alignments, sequence reads generally are aligned to a reference sequence and those that align are designated as being “mapped” or a “sequence tag.” In certain embodiments, a mapped sequence read is referred to as a “hit” or a “count”.

As used herein, the terms “aligned”, “alignment”, or “aligning” refer to two or more nucleic acid sequences that can be identified as a match (e.g., 100% identity) or partial match. Alignments can be done manually or by a computer algorithm, examples including the Efficient Local Alignment of Nucleotide Data (ELAND) computer program distributed as part of the Illumina Genomics Analysis pipeline. The alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand. In certain embodiments, a nucleic acid sequence is aligned with the reverse complement of another nucleic acid sequence.

Various computational methods can be used to map each sequence read to a genomic region. Non-limiting examples of computer algorithms that can be used to align sequences include, without limitation, BLAST, BLITZ, FASTA, BOWTIE 1, BOWTIE 2, ELAND, MAQ, PROBEMATCH, SOAP or SEQMAP, or variations thereof or combinations thereof. In some embodiments, sequence reads can be aligned with sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database. Search hits can then be used to sort the identified sequences into appropriate genomic sections, for example. Some of the methods of analyzing sequence reads are described e.g., US20130288244A1, which is incorporated herein by reference in its entirety.

### Reference Samples

In one aspect, the disclosure provides methods to prepare circulating tumor DNA reference samples. In some embodiment, the methods comprise: inducing apoptosis in cells (e.g., tumor cells) to obtain DNA fragments; and extracting DNA from tumor cells after apoptosis induction to obtain the circulating tumor DNA reference sample. DNA prepared by this method can be used as a reference sample for cfDNA. Moreover, the methods described herein have many advantages, e.g., simple to prepare, short production cycle, low-cost, suitable for many tumor cell types and for large-scale production. The reference samples prepared by the methods described herein can be widely used for methodological validation, internal quality control and external quality evaluation with good reproducibility and consistency.

In some embodiments, cell apoptosis treatment comprises: adding an apoptosis inducer into the tumor cell culture medium, wherein the apoptosis inducer comprises one type that binds to topoisomerase-DNA complex during DNA replication to prevent DNA strand reassembly and cause DNA double strand break.

In some embodiments, the incubation time of the apoptosis inducer is from 2 to 8 hours. Experiments described herein showed that the induction treatment time has an effect on the quality of the reference sample. Either too long or too short of the induction treatment time can lead to deviations in the mutational information of the reference sample, resulting in reduced consistency with the simulated cfDNA and failure to be a good reference. In some embodiments, the incubation time of the apoptosis inducer is about 2-8 hours, about 2-7 hours, about 2-6 hours, about 2-5 hours, about 2-4 hours, about 2-3 hours, about 3-8 hours, about 3-7 hours, about 3-6 hours, about 3-5 hours, about 3-4 hours, about 4-8 hours, about 4-7 hours, about 4-6 hours, about 4-5 hours, about 5-8 hours, about 5-7 hours, about 5-6 hours, about 6-8 hours, about 6-7 hours, or about 7-8 hours. In some embodiments, the incubation time of the apoptosis inducer is about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, or about 8 hours. In some embodiments, the incubation time of the apoptosis inducer is about 2-20 hours, about 2-18 hours, about 2-16 hours, about 2-14 hours, about 2-12 hours, or about 2-10 hours. In some embodiments, the incubation time of the apoptosis inducer is less than 20 hours, less than 18 hours, less than 16 hours, less than 14 hours, less than 12 hours, less than 10 hours, or less than 8 hours, In some embodiments, the incubation time of the apoptosis inducer is at least 30 minutes, at least 1 hour, at least 1.5 hours, at least 2 hours, at least 2.5 hours, at least 3 hours, at least 3.5 hours, at least 4 hours, at least 4.5 hours, or at least 5 hours. In some embodiments, the incubation time of the apoptosis inducer is about 5 hours.

In some embodiments, the concentration of the apoptosis inducer used for treating tumor cells is about 1-100 μM, e.g., about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM. In some embodiments, the concentration of the apoptosis inducer is about 1-50 μM, about 1-40 μM, about 1-30 μM, about 1-20 μM, about 1-10 μM, about 5-50 μM, about 5-40 μM, about 5-30 μM, about 5-20 μM, about 5-10 μM, about 10-50 μM, about 10-40 μM, about 10-30 μM, about 10-20 μM, about 15-50 μM, about 15-40 μM, about 15-30 μM, about 15-20 μM, about 20-50 μM, about 20-40 μM, about 20-30 μM, about 30-50 μM, about 30-40 μM, or about 40-50 μM. In some embodiments, the concentration of the apoptosis inducer is about 5-15 μM, about 5-14 μM, about 5-13 μM, about 5-12 μM, about 5-11 μM, about 5-10 μM, about 5-9 μM, about 5-8 μM, about 5-7 μM, about 5-6 μM, about 6-15 μM, about 6-14 μM, about 6-13 μM, about 6-12 μM, about 6-11 μM, about 6-10 μM, about 6-9 μM, about 6-8 μM, about 6-7 μM, about 7-15 μM, about 7-14 μM, about 7-13 μM, about 7-12 μM, about 7-11 μM, about 7-10 μM, about 7-9 μM, about 7-8 μM, about 8-15 μM, about 8-14 μM, about 8-13 μM, about 8-12 μM, about 8-11 μM, about 8-10 μM, about 8-9 μM, about 9-15 μM, about 9-14 μM, about 9-13 μM, about 9-12 μM, about 9-11 μM, about 9-10 μM, about 10-15 μM, about 10-14 μM, about 10-13 μM, about 10-12 μM, about 10-11 μM, about 11-15 μM, about 11-14 μM, about 11-13 μM, about 11-12 μM, about 12-15 μM, about 12-14 μM, about 12-13 μM, about 13-15 μM, about 13-14 μM, or about 14-15 μM.

In some embodiments, the apoptosis inducer described herein is selected from CPT (Camptothecin), As₂O₃, Notopterol and Gracillin. In some embodiments, the apoptosis inducer described herein can bind to the topoisomerase-DNA complex during DNA replication, e.g., to prevent DNA strand reassembly and/or cause DNA double strand break. In some embodiments, the apoptosis inducer described herein is selected from a raltitrexed or equivalent, or TOMUDEX™; a doxorubicin or equivalent, or ADRIAMYCIN™; a fluorouracil or 5-fluorouracil or equivalent; a docetaxel or equivalent, or TAXOTERE™; a larotaxel, tesetaxel or ortataxel or equivalent; an epothilone or an epothilone A, B, C, D, E or F or equivalent; an ixabepilone (also known as azaepothilone B) or equivalent, or BMS-247550™; a vincristine (also known as leurocristine) or equivalent, or ONCOVIN™; a vinblastin, vinblastine, vindesine, vinflunine, vinorelbine or NAVELBINE™ or equivalent; or, any combination thereof.

In some embodiments, the apoptosis inducer or apoptosis-inducing agent described herein is selected from ABBV-621/APG880, APG350, RG7386/RO6874813, TAS266, MEDI3039, HexaBody®-DR5/DR5 (GEN1029), CPT, and ONC201. Additional apoptosis inducers can be found, e.g., in Lim, B., et al. “Novel apoptosis-inducing agents for the treatment of cancer, a new arsenal in the toolbox.” Cancers 11.8 (2019): 1087; and Fischer, U., et al. “Apoptosis-based therapies and drug targets.” Cell Death & Differentiation 12.1 (2005): 942-961; US20170196901A1; each of which is incorporated herein by reference in its entirety.

In some embodiments, the apoptosis inducer is CPT. Experiments described herein showed that CPT has a better effect and can be adapted to a wide range of tumor cells with broad spectrum.

In some embodiments, the concentration of apoptosis inducer is about 5 to about 15 μM. Experiments described herein showed that the apoptosis inducer does not change the mutation information (e.g., genetic variations) of intracellular DNA, e.g., point mutations, copy number variations, structural variations, fragmentation pattern variations, and other chromosomal variations known in the art, and it can be used to simulate cfDNA. Experiments described herein showed that the DNA breaks induced by the methods described herein can form products similar to nucleosome monomers and their complexes (e.g., double or triple nucleosomal packaging). The fragmented DNA can well mimic the pattern of cfDNA fragmentation compared to methods such as ultrasound treatment, all-trans retinoic acid induction, serum starvation treatment, or intensive culture (e.g., high-density culture) of tumor cells.

In some embodiments, the concentration of apoptosis inducer is about 8-12 μM (e.g., about 10 μM). Experiments described herein showed that the concentration of the apoptosis inducer has an effect on the quality of the reference sample. A too high or too low concentration of the apoptosis inducer can lead to deviations in the mutational information of the reference sample, resulting in the DNA fragments being too small or too large and making them different from those of the simulated cfDNA.

In another aspect, the present disclosure relates to methods of making circulating tumor DNA reference samples. In some embodiments, the methods include: treating tumor cells with CPT to induce cells apoptosis, and extracting DNA from the tumor cells to obtain the circulating tumor DNA reference sample. In some embodiments, the treatment time is about 5 hours and the concentration of CPT is about 10 μM. DNA prepared by the methods described herein can simulate the real plasma cfDNA fragmentation pattern a great extent (e.g., the percentage of each peak representing the nucleosome monomer and their complexes; the DNA fragment size of each peak; the cfDNA unique minor sub-peaks (peaks corresponding to cfDNAs of less than 150 bp); and the ratio between the DNA fragment peaks) and does not affect the detection of point mutations, copy number variations, structural variations, and/or fragmentation pattern variations. DNA prepared by the methods described herein can be used as reference samples for point mutations, copy number variations, structural variations, and DNA fragmentation size detection. In some embodiments, the methods describe herein are easy to operate, have a short synthesis cycle, and can be produced in large-scale (e.g., the percentage of each peak representing the nucleosome monomer and their complexes; the DNA fragment size of each peak; the cfDNA unique minor sub-peaks (peaks corresponding to cfDNAs of less than 150 bp); and the ratio between the DNA fragment peaks).

In another aspect, the disclosure relates to a circulating tumor DNA reference sample, which is prepared by the methods described herein. The products can be used as reference samples for detecting point mutations, copy number variations, structural variations, and DNA fragmentation size detection. In some embodiments, the products can simulate the real plasma cfDNA fragmentation pattern to a great extent.

### Methods of Validating Assays

In another aspect, the disclosure relates to methods for assessing whether the quality of a circulating tumor DNA reference sample is up-to-standard. In some embodiments, the method comprises: (1) extracting DNA from tumor cells that are not treated with an apoptosis inducer, and constructing a DNA library to obtain sequencing reads; (2) constructing a DNA library by sequencing the circulating tumor DNA reference sample described herein; (3) identifying one or more genetic variations of the untreated tumor cells in (1) and one or more genetic variations of the circulating tumor DNA reference sample in (2); and (4) comparing the genetic variations of the untreated tumor cells in (1) and the genetic variations of the circulating tumor DNA reference sample in (2). In some embodiments, a high consistency (e.g., the correlation coefficient R² above 0.5, above 0.55, above 0.6, above 0.65, above 0.7, above 0.75, above 0.8, above 0.85, above 0.9, above 0.91, above 0.92, above 0.93, above 0.94, above 0.95, above 0.96, above 0.97, above 0.98, or above 0.99) of the genetic variations of the untreated tumor cells in (1) and the genetic variations of the circulating tumor DNA reference sample in (2) indicates a good quality of the circulating tumor DNA reference sample. In some embodiments, the one or more genetic variations described herein includes at least one of the following: single nucleotide variations, structural variations, copy number variations and fragmentation pattern variations. In some embodiments, the tumor cells used in (1) and the tumor cells used for making the circulating tumor DNA reference sample described herein are from the same cell line or subject (e.g., human patient). In some embodiments, the tumor cells used in (1) and the tumor cells used for making the circulating tumor DNA reference sample described herein are different.

In some embodiments, prior to comparing the genetic variations of the untreated tumor cells and the circulating tumor DNA reference sample, the methods described herein further include determining and comparing the size distribution of the circulating tumor DNA reference sample and the size distribution of the cell-free DNA(cfDNA) from plasma of a subject (e.g., a human patient described herein), wherein the fragmentation pattern of the size distribution of the circulating tumor DNA reference sample and the size distribution of the cfDNA are similar. For example, the two size distributions can share a fragmentation pattern having one or more of the following patterns: (1) the fragmentation pattern comprises a main peak representing nucleosome monomers (e.g., having a length of about 166 bp); (2) the fragmentation pattern comprises one or more sub-peaks representing complexes of nucleosome monomers (e.g., dimers and trimers); and (3) the fragmentation pattern comprises one or more minor sub-peaks with a length of less than 150 bp. In some embodiments, the nucleosome monomer described herein has a length of about 100-200 bp, about 120-200 bp, about 140-200 bp, about 100-180 bp, about 120-180 bp, about 140-180 bp, about 150-180 bp, about 160-180 bp, or about 160-170 bp. In some embodiments, the nucleosome monomer described herein has a length of about 155 bp, about 158 bp, about 160 bp, about 162 bp, about 164 bp, about 165 bp, about 166 bp, about 167 bp, about 168 bp, about 170 bp, about 172 bp, about 175 bp, or about 180 bp. In some embodiments, the complex of nucleosome monomers has a length of about 320-350 bp (dimer), about 480-510 bp (trimer). In some embodiments, the complex of nucleosome monomers has a length that is about 1-fold, 2-fold, 3-fold, 4-fold, 5-fold, 6-fold, 7-fold, or 8-fold of the length of a nucleosome monomer described herein. In some embodiments, the minor sub-peaks described herein have a length of about 50-166 bp, about 50-160 bp, about 50-150 bp, about 50-140 bp, about 60-166 bp, about 60-160 bp, about 60-150 bp, or about 60-140 bp. In some embodiments, the size distribution described herein includes at least 1, at least 2, at least 3, at least 4, at least 5, at least 6, at least 7, at least 8, at least 9, or at least 10 minor sub-peaks with a length less than 150 bp.

In some embodiments, the disclosure described herein provides methods of determining the limit of detection (LOD) of mutation frequency of an assay. In some embodiments, the methods involve mixing different samples' DNA at different ratios to obtain a series of DNA samples. In some embodiments, the methods involve preparing a standard curve to detect LOD, which is generally known in the art.

In some embodiments, the disclosure described herein provides methods of determining other dimensions of an assay, e.g., repeatability, reproducibility, positive and negative percentage agreement.

### Methods of Predicting Cancer

In another aspect, the disclosure relates to the use of the above methods and circulating tumor DNA reference samples in constructing a tumor prediction model. In some embodiments, the circulating tumor DNA reference prepared by the methods described herein can be used for testing the performance of the cancer prediction model. For example, construction of a pan-cancer early screening method or model. The cfDNA fragment profile of normal individuals is more stable, while the cfDNA fragment profile of cancer patients is relatively heterogeneous, and there is a significant difference between the two. For example, the MCRS model disclosed in U.S. Patent Application Publication No. 20220136062A1, which is incorporated herein by reference in its entirety, is based on comparing the distribution of copy number variation (CNV), fragment size (FS) and protein markers between normal and cancer patients, and standardizing all the quantified dimensions. Finally, the cancer contribution of each standardized dimension is weighted to obtain the overall cancer risk score (CRS). Using the reference sample from the disclosure herein to measure the performance of the model for single nucleotide variations (SNV), structural variation (SV), copy number variation (CNV) and fragment size detection. The reference samples as descried herein can be used to complete the whole process through method development, optimization, and performance confirmation to validate the real performance of the model before it is applied to the clinic.

In some embodiments, the cancer described herein is a blood cancer (e.g., leukemia or lymphoma). In some embodiments, the cancer described herein is a solid tumor. In some embodiments, the cancer described herein is any cancer type that can be potentially diagnosed from cfDNA.

In some embodiments, the disclosure relates to methods of predicting cancer, including (1) determining the size distribution of the cell-free DNA (cfDNA) from plasma of a subject; (2) determining the size distribution of the circulating tumor DNA reference sample of claims 11; and (3) comparing the size distribution of the cfDNA in (1) and the size distribution of the circulating tumor DNA reference sample in (2). In some embodiments, a matching fragmentation pattern of the size distribution of the cfDNA in step (1) and the size distribution of the circulating tumor DNA reference sample in step (2) indicates existence of cancer in the subject. In some embodiments, the fragmentation pattern is matched when the Pearson correlation coefficient is at least 0.3, a least 0.35, at least 0.4, at least 0.45, at least 0.5, at least 0.55, at least 0.6, at least 0.65, or at least 0.7 for fragments less than 166 bp (e.g., between 50-166 bp, between 60-166 bp, between 70-166 bp, between 80-166 bp, between 90-166 bp, or between 100-166 bp) between the size distribution of the cfDNA in step (1) and the size distribution of the circulating tumor DNA reference sample in step (2).

### Kits

In another aspect, the disclosure relates to the application of the above reference sample in a kit for cancer prediction. The reference products prepared using the methods described herein are used in kits for cancer prediction. Laboratories performing internal quality control and third-party organizations performing external quality evaluation and proficiency testing need reference samples to ensure the reliability of test results. The reference samples described herein can realize standardized high-throughput sequencing assays, perform performance confirmation or performance validation, internal quality control, and external quality evaluation.

In some embodiments, the samples are derived from tissues, blood, or tumor cells of animals (e.g., common experimental animals including mice, rats, guinea pigs, hamsters, rabbits, dogs, monkeys, pigs, fish and so on).

In another aspect, the disclosure relates to a kit for cancer screening, which contains a circulating tumor DNA reference sample described herein.

Additional aspects and advantages of the present invention will be given in part of the following description, and will become apparent from the following description, or known through the practice of the invention.

All numeric values in the disclosure are herein assumed to be modified by the term “about”, whether or not explicitly indicated. As used herein, the term “about” generally refers to a range of numbers that one of skill in the art would consider equivalent to the recited value (i.e., having the same function or result). In some embodiments, the terms “about” may include numbers that are rounded to the nearest significant figure. In some embodiments, the terms “about” may include numbers that are ±10%, ±20%, or ±30% of the value.

In the description of this specification, references to the terms “one embodiment”, “some embodiments”, “examples”, “concrete examples”, or “some examples”, etc. mean that the specific features, structures, materials, or features described in combination with such embodiments or examples are contained in at least one embodiment or example of the invention. In this specification, indicative representations of the above terms do not need refer to the same embodiments or examples. Furthermore, the specific features, structures, materials or features described may be combined in an appropriate manner in any one or more embodiments or examples. In addition, in the case of non-conflict, technicians in the field may combine together the different embodiments or examples described in this specification or the characteristics of the different embodiments or examples.

### EXAMPLES

The invention is further described in the following examples, which do not limit the scope of the invention described in the claims.

### Example 1: Methods and Materials

### Cell Culture and Induction of Apoptosis

Cell culture medium was prepared by mixing 20% fetal bovine serum (FBS) and 80% Iscove's Modified Dulbecco's Medium (IMDM). The culture medium was supplemented with Gentamicin (Gentamicin dosage: 40,000 units/500 mL cell culture medium).

Camptothecin (CPT) was dissolved in DMSO to 4 mg/mL (maximal solubility: 5 mg/mL) and diluted to 1 mg/mL when used. The CPT solution was filter sterilized with a 0.22 μm needle filter, aliquoted into 1.5 mL centrifuge tubes, and then stored in a refrigerator at 4° C. As DMSO solidifies at 4° C., the tubes containing CPT were thawed prior to subsequent experiments.

2-4×10⁶ HL-60 resistance cells or NB-4 cells were cultured overnight in 75 cm² culture flasks supplemented with 20 mL complete medium and placed in a 37° C./5% CO₂ incubator to ensure cells entered logarithmic growth phase. The cells were treated with apoptosis inducers (e.g.,

CPT and ARTA) or cultured with high-density separately as follows. (1) After the cells were cultured overnight, old culture medium was carefully removed and discarded. 20 mL fresh culture medium and 69.67 μl CPT solution were added to make the final concentration 10 μM, and the cells were collected after an incubation for 5-24 hours. (2) All trans-retinoic acid (ATRA): After the cells were cultured overnight, old culture medium was carefully removed and discarded. 20 mL fresh culture medium and ATRA were added to make the final concentration 10 μM, and the cells were collected after an incubation for 3 days. (3) After cells were cultured overnight, the cells were let grow without changing the culture medium or passaging.

The cells suspended in the culture medium were harvested, transferred into tubes, and centrifuged at 800 rpm for 7 minutes. Cells were pelleted at tube bottom and collected for subsequent experiments.

Extraction of DNA from Apoptotic Cells

Apoptotic DNA Ladder Extraction Kit (Beyotime, Cat#: C0008) was used. Specifically, 200 μL of PBS was added to the collected cell pellets and gently pipetted to resuspend the cells in PBS. 4 μL RNase A was added and mixed thoroughly by vortexing. The cell suspension was kept at room temperature (15-25° C.) for 3-5 minutes. Afterwards, 20 μL Proteinase K was added and mixed thoroughly by vortexing. 200 μL Lysis Buffer B was then added and mixed thoroughly by vortexing. The cell lysate was incubated at 70° C. for 10 minutes. After the incubation, 200 μL ethanol (96-100%) was added and mixed thoroughly by vortexing. The mixture from the previous step was added to a DNA purification column, which was centrifuged at 6000×g (about 8000 rpm) for 1 minute. Liquid waste was discarded. 500 μL of Washing Solution I was then added and the column was centrifuged at 6000×g (about 8000 rpm) for 1 minute. Liquid waste was discarded. Next, 600 μL of Washing Solution II was added and the column was centrifuged at 18,000×g (about 12,000 rpm) for 1 minute. Liquid waste was discarded. Next, the column was centrifuged at 18,000×g (about 12,000 rpm) for 1 minute to remove residual ethanol. The column was then placed in a clean 1.5 mL elution tube and 50 μL of elution buffer was carefully applied. The tube was centrifuged at 12,000 rpm for 1 minute to elute the total DNA.

1 μL of the obtained total DNA was subjected to quantification by the Qubit™ fluorometer. Another 1 μL of the obtained total DNA was used for fragment size detection using the Agilent 2100 Bioanalyzer. If apoptosis occurred, a typical DNA ladder can be observed. Because of apoptosis-induced fragmentation, the obtained DNA was subjected to library construction without shearing.

DNA Library Construction KAPA Hyper Prep Kit (Kapa Biosystems, Cat#: KK8504) was used to construct DNA libraries.

End Repair and A-Tailing

Each end repair and A-tailing reaction was prepared in a tube or a well of a PCR plate as shown in the table below.

TABLE 1

| Component | Volume |
|---|---|
| Fragmented, double-stranded DNA | 50 μL (~50 ng) |
| End Repair & A-Tailing Buffer | 7 μL |
| End Repair & A-Tailing enzyme mix | 3 μL |
| Total volume | 60 μL |

The reaction system was mixed gently by vortexing, spun down briefly, and then kept on ice. Immediately afterwards, the tube/plate was placed in a thermocycler programmed as shown in the table below.

TABLE 2

| Step | Temperature | Time |
|---|---|---|
| End Repair and A-Tailing | 20° C. | 30 min |
| | 65° C. | 30 min |
| HOLD | 4° C. | ∞ |

Adapter Ligation

In the same tube/plate where end repair and A-tailing was performed, the following adapter ligation reaction was prepared.

TABLE 3

| Component | Volume |
|---|---|
| End Repair and A-Tailing reaction product | 60 μL |
| Adapter stock (15 μM) | 5 μL |
| PCR-grade water | 5 μL |
| Ligation Buffer | 30 μL |
| DNA Ligase | 10 μL |
| Total volume | 110 μL |

The reaction system was mixed thoroughly, centrifuged briefly, and then incubated at 20° C. for 15 minutes.

Post-Ligation Cleanup

80% ethanol (e.g., 50 mL of 80% ethanol can be prepared by mixing 40 mL of absolute ethanol and 10 mL of nuclease-free water) was prepared before use. 1.5 mL centrifuge tubes were prepared and labeled with the corresponding number. Magnetic beads that had been pre-equilibrated at room temperature were fully vortexed and mixed. Each tube was filled with 88 μL of the magnetic beads.

The above DNA mixture was mixed with the magnetic beads, and incubated at room temperature for 10 minutes. After the incubation, the 1.5 mL tubes were placed on the magnet to capture the magnetic beads until the liquid became clear. Supernatant was carefully removed and discarded. 200 μL of 80% ethanol was added into each tube. The tubes were rotated 360 degrees horizontally and incubated on the magnet at room temperature for 30 seconds. Afterwards, supernatant was discarded while the tubes were kept on the magnet.

The above steps were repeated once. Afterwards, all residual ethanol was removed without disturbing the beads. The tube cap was opened to dry the magnetic beads at room temperature and volatilize the ethanol. Residual ethanol can negatively affect the enzymatic function of the enzymes used in the subsequent reaction systems. However, the magnetic beads should not be excessively dried, otherwise the DNA cannot be easily eluted from the magnetic beads, resulting in reduced yield. The drying was stopped once the surface of the magnetic beads was no longer shiny.

21 μL of nuclease-free water was added into each centrifuge tube to resuspend the magnetic beads. After thorough mixing, the tubes were incubated at room temperature for 5 minutes. A new batch of 200 μL PCR tubes were prepared and labeled. The tubes were placed on the magnet to capture the magnetic beads until the solution was clear, then the supernatant was transferred to the corresponding PCR tube as a template for the PCR experiment.

Library Amplification

The library amplification reaction system was prepared as follow:

TABLE 4

| Component | Volume |
|---|---|
| 2 × KAPA HiFi Hotstart Ready Mix | 25 μL |
| 10 × KAPA Library Amplification Primer mix | 5 μL |
| Total master mix volume | 30 μL |

30 μL of pre-PCR amplification reaction system was added to each 0.2 mL PCR tube, mixed gently and centrifuged at low speed. Afterwards, the PCR tubes were placed in a thermocycler programed as shown in the table below.

TABLE 5

| Step | Temperature | Reaction time | | Cycle number |
|---|---|---|---|---|
| Preliminary | 98° C. | 45 | s | 1 |
| denaturation | | | | |
| Denaturation | 98° C. | 15 | s | 4 |
| Annealing | 60° C. | 30 | s | |
| Elongation | 72° C. | 30 | s | |
| Final elongation | 72° C. | 1 | min | 1 |

| | | | |
|---|---|---|---|
| Storage | 4° C. | ∞ | 1 |

After the pre-PCR reaction was finished, the library was purified as described below.

Post-Amplification Purification

1.5 mL sample tubes were prepared and labeled with the corresponding numbers. Magnetic beads that had been pre-equilibrated at room temperature were fully vortexed and mixed. Each tube was filled with 50 μL of the magnetic beads. The above DNA mixture was mixed with the magnetic beads, and incubated at room temperature for 10 minutes. After the incubation, the 1.5 mL tubes were placed on the magnet to capture the magnetic beads until the liquid became clear. Supernatant was carefully removed and discarded. 200 μL of 80% ethanol was added into each tube. The tubes were rotated 360 degrees horizontally and incubated on the magnet at room temperature for 30 seconds. Afterwards, supernatant was discarded while the tubes were kept on the magnet.

The above steps are repeated once. Afterwards, all residual ethanol was removed without disturbing the beads. The tube cap was opened to dry the magnetic beads at room temperature and volatilize the ethanol. Residual ethanol can negatively affect the enzymatic function of the enzymes used in the subsequent reaction systems. However, the magnetic beads should not be excessively dried, otherwise the DNA cannot be easily eluted from the magnetic beads, resulting in reduced yield. The drying was stopped once the surface of the magnetic beads was no longer shiny.

35 μL of nuclease-free water was added to each sample tube to resuspend the magnetic beads. After thorough mixing, the tubes were incubated at room temperature for 5 minutes. A new batch of PCR tubes were prepared and labeled. The tubes were placed on the magnet to capture the magnetic beads until the solution was clear, then the supernatant was transferred to new 1.5 mL tubes labeled with sample information.

Quality Control

1 μL of the obtained total DNA was subjected to quantification by the Qubit™ fluorometer. Another 1 μL of the obtained total DNA was used for fragment size detection using the Agilent 2100 Bioanalyzer.

### Genomic DNA Fragmentation by Ultrasonication

DNA fragmentation was performed using the Covaris® M220 non-contact ultrasonic fragmentation instrument. Specifically, a power-up check was performed as follows: (1) the computer fixed on the top of the instrument was properly wired to the machine; and (2) the Drip Tray was placed under the machine; and (3) the operating tube holder was inserted. The power of instrument and computer were turned on, and the controlling software was clicked open to make the system in operational mode. The sliding weight on the top of the tube holder was pulled up and rotated by 90 degrees. Approximately 15 mL of distilled or deionized water was added into the center of the holder. The water level should reach the green “√” status or exceed the “RUN” marker, and the water level just touched the operating tube holder completely.

1 μg genomic DNA was pipetted into a 1.5 mL tube and 1×Low TE Buffer was added to make the volume 50 μL. The diluted genomic DNA sample was mixed gently and transferred carefully to the ultrasonication tube to avoid bubbles. The sliding weight on the top of the tube holder was pulled up and rotated by 90 degrees.

The ultrasonication tube with sample was placed into the instrument. The sliding weight was rotated and pushed down so that it pressed against the sample tube. The safety gate was then closed. The program used is shown in the table below.

TABLE 6

| Setting | Reference value |
|---|---|
| Max. incident power (W) | 75 |
| Working factor (%) | 10 |
| Number of ultrasonic | 200 |
| energy transfer (cpb) | |
| Processing time(sec) | 210 |

Next, the “Run” button was clicked in the RUN interface to run the program. When the program was finished, the water bath was emptied with a syringe. Residual water in the Drip Tray was also emptied and dried with dustless paper. The software, the instrument, and the computer were then closed in order.

The following procedures were also performed with caution. (1) The room temperature of the laboratory was kept at 15-30° C., and not too cold. (2) The program was run with a water bath to avoid damaging the sensor. (3) Only double-distilled water or deionized water was used for the water bath. (4) At the end of daily use, the Drip Tray was emptied and dried to prevent growth of microorganisms. (5) The safety door was closed when operating the system. (6) The DNA fragments were temporarily stored at −20° C.

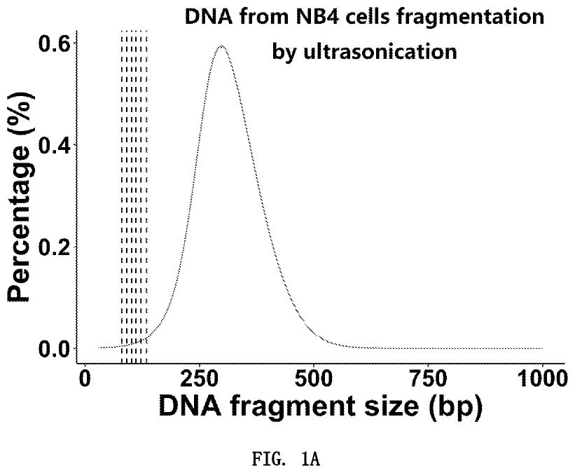

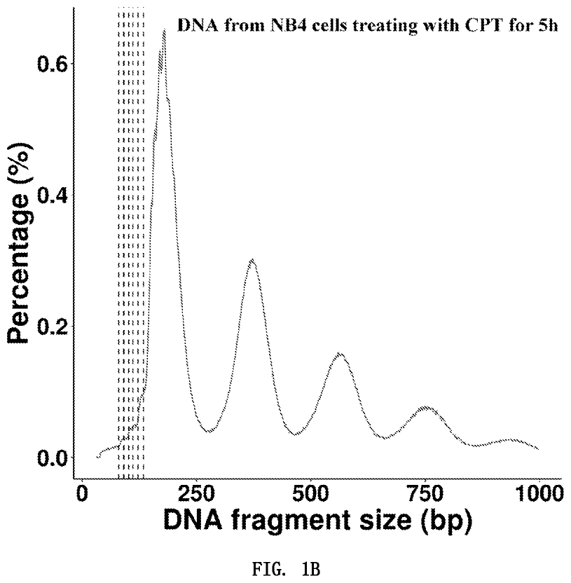

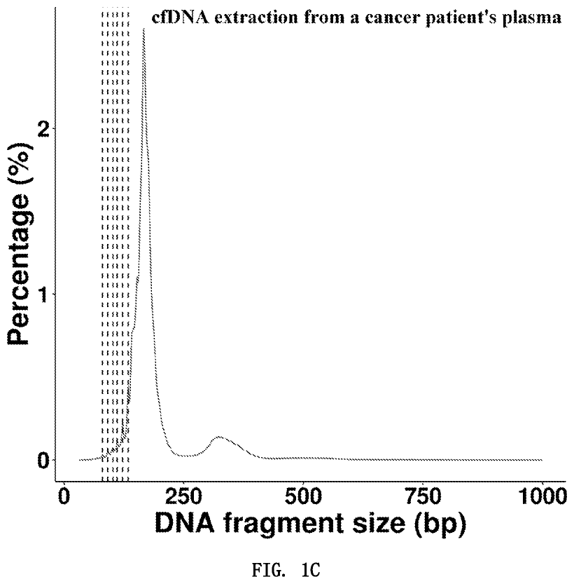

The distribution of the genomic DNA fragmented by ultrasonication as described above was compared with the distribution of plasma cfDNA fragments. The results are shown in FIG. 1A and FIG. 1C, respectively, and discussed in Example 2.

Circulating-Free DNA (cfDNA) Extraction from Plasma

The equipment, reagents, and consumables required for the experiments below were prepared. A water bath was switched on and the temperature was adjusted to 60° C. A heating block was switched on and the temperature was adjusted to 56° C. Extraction was performed using the QIAamp® Circulating Nucleic Acid Kit (Qiagen, Cat#: 55114). Buffers and reagents (Buffer ACB, Buffer ACW1, Buffer ACW2, ACL mixture, and carrier RNA dissolved in Buffer ACL) were prepared per the manufacturer's instructions.

Lysis of Plasma

400 μl Proteinase K was pipetted into a 50 mL centrifuge tube, and 4 ml plasma was added to the 50 mL tube. 3.2 ml Buffer ACL (containing 1.0 μg carrier RNA) was then added. The tube cap was closed and the solution was mixed by pulse-vortexing for 30 seconds, with an observable vortex formed in the tube. To ensure efficient lysis, the sample and Buffer ACL were mixed thoroughly to yield a homogeneous solution. The sample was incubated at 60° C. for 30 minutes immediately after the mixing step. After incubation, 7.2 mL Buffer ACB was added to the lysate in the tube. The tube cap was closed and the solution was mixed thoroughly by pulse-vortexing for 15 seconds. The lysate-Buffer ACB mixture was incubated in the tube for 5 minutes on ice or in a refrigerator.

Assembly of the Suction Filtration Device

The QIAvac 24 Plus system was connected to a vacuum source. A VacValve was inserted into each luer slot of the QIAvac 24 Plus. A VacConnector was inserted into each VacValve. The QIAamp Mini columns were inserted into the VacConnectors on the manifold. Finally, a tube extender (20 mL) was inserted into each QIAamp Mini column. The tube extender was firmly inserted into the QIAamp Mini column to avoid leakage of sample. More specifically, the 2 mL collection tube was remained for the subsequent operation. The sample number was marked on the QIAamp Mini silica membrane column. VacValve ensured a steady flow rate. VacConnectors prevented direct contact between the spin column and VacValve during purification, thereby avoiding any cross-contamination between samples. The QIAamp Mini silica membrane column can adsorb DNA, and the tube extender can hold large volumes of plasma.

DNA Purification and Elution

The lysate-Buffer ACB mixture was carefully applied to the tube extender of the QIAamp Mini column. The vacuum pump was switched on. When all lysates had been drawn through the columns completely, the vacuum pump was switched off and the exhaust valve was opened to release the pressure to 0 mbar. The tube extender was carefully removed and discarded. 600 μL Buffer ACW1 was applied to the QIAamp Mini column. The exhaust valve was closed and the vacuum pump was switched on. After all of Buffer ACW1 had been drawn through the QIAamp Mini column, the vacuum pump was switched off and the exhaust valve was opened to release the pressure to 0 mbar. 750 μL Buffer ACW2 was applied to the QIAamp Mini column. The exhaust valve was closed and the vacuum pump was switched on. After all of Buffer ACW2 had been drawn through the QIAamp Mini column, the vacuum pump was switched off and the exhaust valve was opened to release the pressure to 0 mbar. 750 μL ethanol (96-100%) was applied to the QIAamp Mini column. The exhaust valve was closed and the vacuum pump was switched on. After all of the ethanol had been drawn through the QIAamp Mini column, the vacuum pump was switched off and the exhaust valve was opened to release the pressure to 0 mbar. The lid of the QIAamp Mini column was closed and removed from the vacuum manifold. The VacConnector was discarded. The QIAamp Mini column was placed in a clean 2 mL collection tube, and centrifuged at full speed (20,000×g; 14,000 rpm) for 3 minutes. The QIAamp Mini Column was placed into a new 2 mL collection tube. The lid was opened, and the assembly was incubated at 56° C. for 10 minutes to dry the membrane completely. The QIAamp Mini column was placed in a clean 1.5 mL elution tube (included in the kit), and the 2 mL collection tube was discarded. 55 μL of nuclease-free water was carefully applied to the center of the QIAamp Mini membrane. The lid was closed and incubated at room temperature for 3 minutes. After the incubation, the tube was centrifuged in a microcentrifuge at full speed (20,000×g; 14,000 rpm) for 1 minute to elute the nucleic acids.

### Example 2: Comparison of DNA Fragment Size Distribution

Different methods were used to prepare fragmented DNA, and the DNA obtained by inducing cell apoptosis was similar to the real cfDNA fragmentation pattern. Specifically, the DNA fragments were obtained by shearing the genomic DNA of NB4 cells to 200-300 bp by ultrasonication, and the DNA fragment size distribution is shown in FIG. 1A. NB4 cells were treated with CPT for 5 hours before DNA extraction, and the distribution of DNA fragment size is shown in FIG. 1B. The distribution of the fragment size of cfDNA obtained from a cancer patient's plasma is shown in FIG. 1C. Some minor sub-peaks (marked by dashed lines) can also be detected in fragments of cfDNA less than 150 bp. The results showed that the minor sub-peaks can also be detected in the reference sample in FIG. 1B.

The results showed that the fragment size distribution of the DNA derived from CPT-induced apoptotic NB4 cells was similar to that of patient-derived cfDNA, both exhibiting multi-peak characteristics related to single, double, or triple nucleosomal packaging, whereas the fragment size distribution of DNA from ultrasound-treated NB4 cells was different from that of patient-derived cfDNA or DNA derived from artificially-induced apoptotic NB4 cells. The results indicate that DNA prepared by inducing cell apoptosis can be used as a reference sample for analyzing the fragmentation pattern of cfDNA.

### Example 3: Optimization of Apoptosis Induction Conditions

The DNA fragments were prepared using different cells and by different apoptosis-inducing methods to find the optimal experimental conditions.

TABLE 7

| Cell types | Methods | | | |
|---|---|---|---|---|
| HL-60 | Treating | Treating | High-density | Treating |
| resistance | with CPT | with ATRA | culture for | with CPT |
| cell | for 5 h | for 3 d | 3 d | for 24 h |
| NB4 | Treating | Treating | High-density | / |
| cell | with CPT | with ATRA | for 3 d | |
| | for 5 h | for 3 d | | |

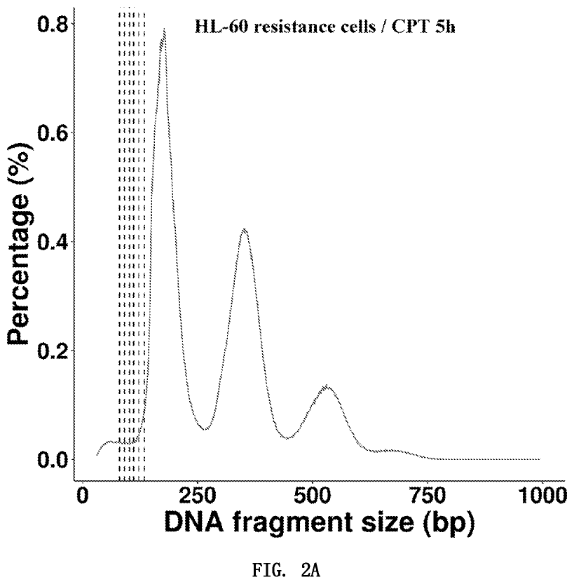

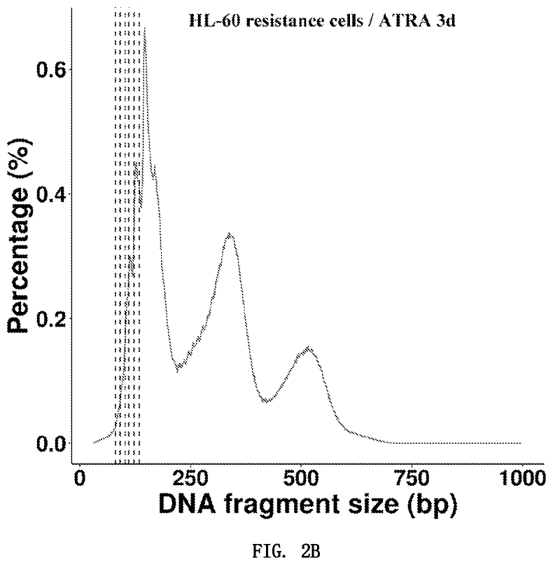

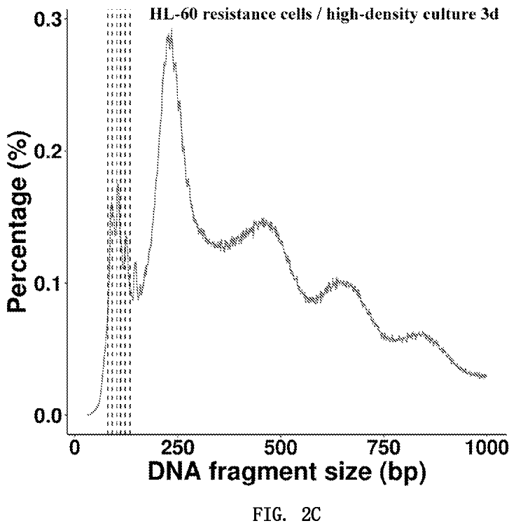

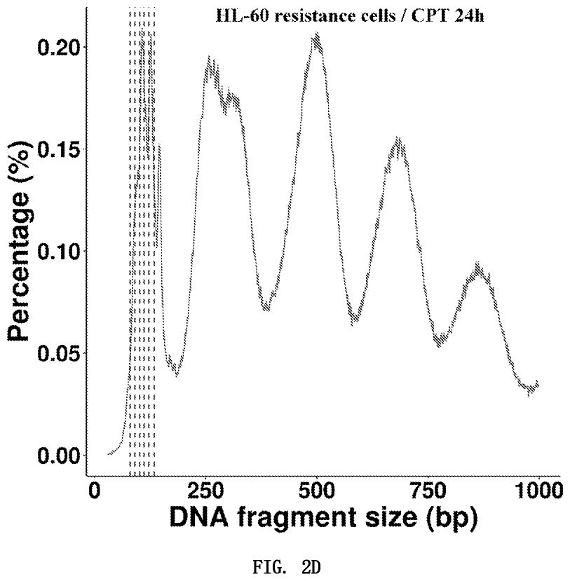

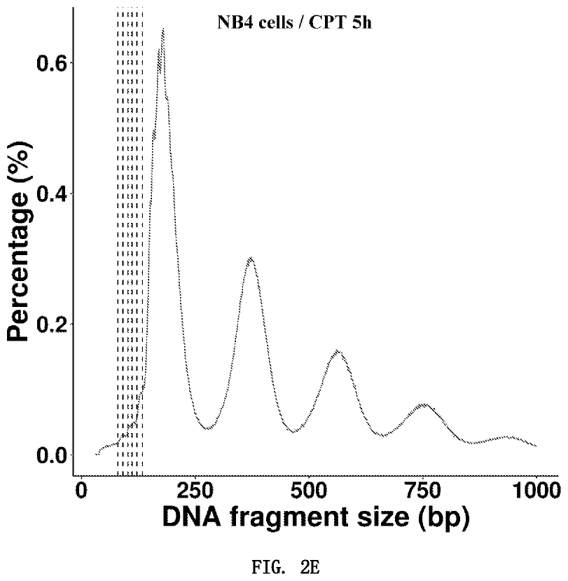

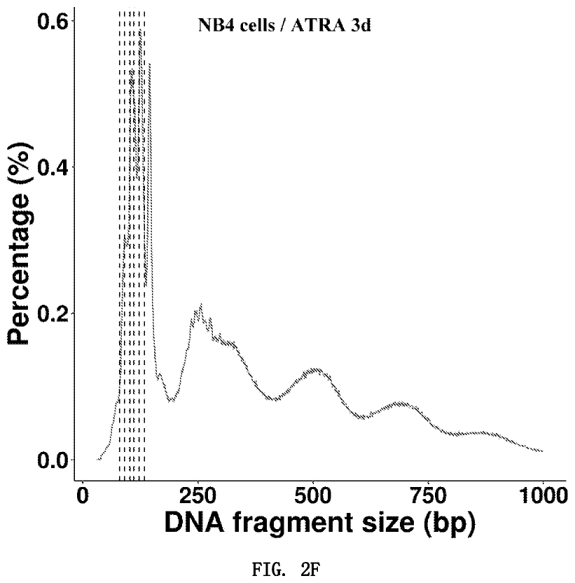

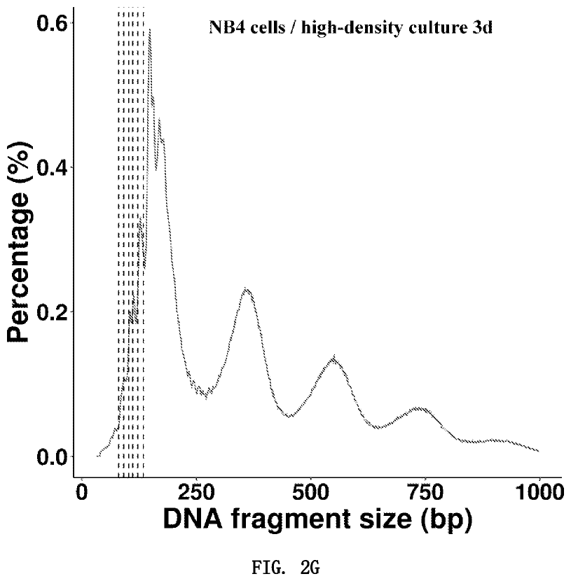

Different apoptosis-inducing methods for HL-60 resistance cells or NB4 cells are listed in Table 7. The distribution of fragment size of DNA extracted after each apoptosis induction are shown in FIGS. 2A-2G.

The results showed that treatment with CPT for 5 hours can be used as an optimal apoptosis-inducing condition for the production of cfDNA reference sample, which yielded DNA fragments with similar fragmentation pattern to patient-derived cfDNA, while the fragmentation patterns of DNA fragments obtained by other apoptosis-inducing methods showed obvious differences from that of patient-derived cfDNA.

### Example 4: Validation of Fragmented DNA from Artificially-Induced Apoptotic Cells

The NB4 cells were separated into two groups. Cells from one group (experimental) were treated with CPT for 5 hours to induce apoptosis before DNA extraction, library construction and sequencing analysis. Cells from the other group (control) without drug treatment were used as controls for DNA extraction, DNA fragmentation by ultrasonication, library construction and sequencing analysis. The libraries of both samples were subjected to whole-genome sequencing at a depth of 50×, and the sequencing data were then compared and analyzed.

The results showed that the DNA obtained from artificially-induced apoptotic cells had high consistency with the DNA from untreated cells in terms of its point mutation, copy number variation, structural variation and other variation information.

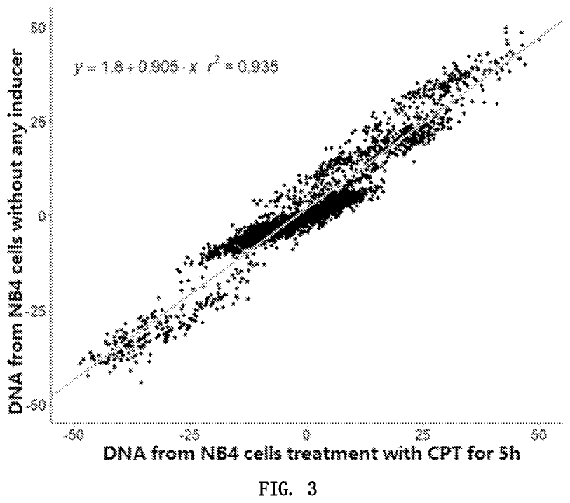

First, the copy number variation of DNA in the experimental and control groups was consistent. Consistency analysis was performed on the copy number variation between the DNA produced by drug-induced apoptosis and the DNA of the control group. The results are shown in FIG. 3. The correlation coefficient R² of the two was determined as 0.935, indicating that the copy number variation of the two samples was highly consistent. The results also suggest that drug-induced apoptosis did not affect the copy number variation.

Second, typical structural variation was detected in both experimental and control groups, e.g., PML/RARA gene fusion. As an acute promyelocytic leukemia cell line, NB4 cells have typical PML/RARA gene fusion variations. The analysis results showed that the fusion gene was detected in both the experimental group and the control group, and the specific results are shown in Table 8, indicating that drug-induced apoptosis did not affect the chromosome structural variation.

TABLE 8

| | | 5′ break | | 3′ break | | Support | Support reads |
|---|---|---|---|---|---|---|---|
| Sample name | Chromosome | site | Chromosome | site | Type | type | (Ref, Alt) |
| Control | Chr15 | 74326370 | Chr17 | 38502180 | Fusion | PR:SR | 26, 11:25, 3 |
| group | Chr17 | 38502180 | Chr15 | 74326370 | Fusion | PR:SR | 26, 11:25, 3 |
| Experimental | Chr15 | 74326370 | Chr17 | 38502180 | Fusion | PR:SR | 28, 6:21, 3 |
| group | Chr17 | 38502180 | Chr15 | 74326370 | Fusion | PR:SR | 28, 6:21, 3 |

PR: pair reads;
SR: splicing reads;
(Ref, Alt): (Number of reads across breakpoints for wild type, number of reads across fusion sites for mutant type)

Finally, a high SNP consistency was observed between experimental and control groups. Specifically, the high-frequency SNPs in the DNA of the experimental group and the control group were compared, and the results showed that the consistency of the SNPs in the experimental group and the control group reached 99.6%, indicating that drug-induced apoptosis did not change the point mutation information in the cell genome.

Therefore, the DNA fragments obtained by the methods of drug-induced apoptosis described herein can simulate the fragmentation pattern of real plasma cfDNA to a great extent, and do not affect the detection of point mutations, copy number variations, structural variations, etc. The DNA fragments prepared by this method can be used as reference samples for point mutation, copy number variation, structural variation, and DNA fragmentation size detection. The methods are easy to operate, have a short synthesis cycle, and are suitable for mass production. Thus, the methods described herein can be widely used in methodological validation, internal quality control and inter-laboratory quality evaluation, with good repeatability and consistency.

### Example 5: Application of the ctDNA Reference Sample

The reference samples described herein can be used as an internal quality control in assays. For example, cancer cell lines with targeted mutation sites can be selected as positive controls, and a normal cell line (e.g., GM12878) can be selected as a negative control. Apoptosis can be induced in cells, and DNA can be extracted from these cells to produce reference samples. The DNA from cancer cells with specific mutations can be used as a positive control, and the DNA from normal cells without mutations can be used as a negative control. These reference samples can be subjected to library preparations, sequencing, and data analyses in the same batch with the experimental samples to monitor the whole process. When the mutations can be detected in the positive reference sample but can't be detected in the negative reference sample, experiments can be proceeded normally with a satisfactory quality control result. However, when the mutations can't be detected in the positive reference sample, experiments should be terminated because of the failure of quality control. Alternatively, when the mutations can be detected in the negative reference sample, experiments should also be terminated because of failure of quality control, or an indication of contamination in the assays.

The reference samples described herein can be used in performance validation. Through sequential dilution, the positive samples and negative samples can be blended to obtain a series of reference samples with a gradient of tumor DNA fractions. These DNA reference samples can be subjected to library preparations, sequencing, and data analyses with experimental samples. The LOD of the assay can be determined by testing reference samples with different mutation frequencies. The repeatability and reproducibility can be determined by repeated testing of these reference samples. The sensitivity and specificity of the assay can be determined by testing a variety of positive and negative references.

Because the reference samples described herein have the fragmentation characteristic of real plasma-derived cfDNA, they can be used as a control to study the fragmentation pattern in the assays. The reference samples described herein can also play an important role in assay development, efficacy tests and other applications.

The DNA reference samples described herein can also be used as a control for the DNA extraction step. For example, the DNA reference samples can be added into artificial plasma to produce plasma reference samples to mimic real human plasma with different DNA mutation frequency. In the DNA extraction experiment, the reference samples can be extracted at the same time, to monitor whether there is contamination in the experiment process and whether there is any problem in the experimental procedure.

### Other Embodiments

It is to be understood that, while the invention has been described in conjunction with the detailed description thereof, the foregoing description is intended to illustrate without limiting the scope of the invention, which is defined by the appended claims. Other aspects, advantages, and modifications are within the scope of the following claims.

1. A method of preparing a circulating tumor DNA reference sample, the method comprising:
(1) inducing apoptosis in tumor cells; and
(2) extracting DNA from the tumor cells to obtain the circulating tumor DNA reference sample.

2. The method of claim 1, wherein the tumor cells are incubated with an apoptosis inducer in a culture medium) to induce apoptosis.

3. The method of claim 2, wherein the apoptosis inducer can bind to the topoisomerase-DNA complex during DNA replication to prevent DNA strand reassembly or cause DNA double strand break.

4. The method of claim 2, wherein the tumor cells are incubated with the apoptosis inducer in the culture medium for 2-8 hours.

5. The method of claim 2, wherein the tumor cells are incubated with the apoptosis inducer in the culture medium for about 5 hours.

6. The method of claim 2, wherein the apoptosis inducer is selected from the group consisting of As₂O₃, notopterol and gracillin.

7. The method of claim 2, wherein the apoptosis inducer is camptothecin (CPT).

8. The method of claim 2, wherein the concentration of the apoptosis inducer is about 5-15 μM.

9. The method of claim 2, wherein the concentration of the apoptosis inducer is about 10 μM.

10. (canceled)

11. A circulating tumor DNA reference sample obtained using the method of claim 1.

12. A method for determining the quality of the circulating tumor DNA reference sample of claim 11, the method comprising:
(1) providing a first DNA library of DNA extracted from tumor cells that are not treated with an apoptosis inducer;
(2) providing a second DNA library by sequencing the circulating tumor DNA reference sample;
(3) identifying one or more genetic variations in the first DNA library and one or more genetic variations in the second DNA library; and
(4) comparing the one or more genetic variations in the first and second DNA libraries;
wherein consistency of the genetic variations in the first and second DNA libraries indicates a good quality of the circulating tumor DNA reference sample.

13. The method of claim 12, wherein the one or more genetic variations are selected from the group consisting of single nucleotide variations, structural variations, copy number variations, and/or fragmentation pattern variations.

14. The method of claim 12, further comprising, prior to step (1): determining and comparing the size distribution of the circulating tumor DNA reference sample and the size distribution of the cell-free DNA (cfDNA) from plasma of a subject, wherein the size distributions of the circulating tumor DNA reference sample and the cfDNA share a fragmentation pattern having one or more of the following features:
(1) the fragmentation pattern comprises a main peak representing nucleosome monomers with a length of about 166 bp;
(2) the fragmentation pattern comprises one or more sub-peaks representing complexes of nucleosome monomers; and
(3) the fragmentation pattern comprises one or more minor sub-peaks with a length of less than 150 bp.

15. A method of predicting cancer using the circulating tumor DNA reference sample of claim 11.

16. A method of predicting cancer, comprising:
(1) determining the size distribution of the cell-free DNA (cfDNA) from plasma of a subject;
(2) determining the size distribution of the circulating tumor DNA reference sample of claims 11; and
(3) comparing the size distribution of the cfDNA in (1) and the size distribution of the circulating tumor DNA reference sample in (2);
wherein a matching fragmentation pattern of the size distribution of the cfDNA in step (1) and the size distribution of the circulating tumor DNA reference sample in step (2) indicates existence of cancer in the subject, wherein the fragmentation pattern is matched when the Pearson correlation coefficient is at least 0.5 for fragments between 50-166 bp between the size distribution of the cfDNA in step (1) and the size distribution of the circulating tumor DNA reference sample in step (2).

17. The method of claim 16, wherein the subject is a human patient diagnosed with cancer, suspected to have cancer, or having a risk to have cancer.

18. (canceled)

19. A method of determining the limit of detection (LOD) of mutation frequency of an assay, comprising:
(1) providing DNA extracted from a first type of cells treated with an apoptosis inducer, wherein the first type of tumor cells have one or more mutations at a chromosomal site;
(2) providing DNA extracted from a second type of cells treated with the apoptosis inducer, wherein the second type of tumor cells have no mutation at the chromosomal site;
(3) mixing the DNA from step (1) and step (2) at different ratios to obtain a series of DNA samples;
(4) constructing one or more DNA libraries from the series of DNA samples; and
(5) determining the frequency of the one or more mutations from the constructed DNA libraries,
wherein the LOD of mutation frequency of the assay can be determined by the frequency of the one or more mutations from the constructed DNA libraries.

20.-24. (canceled)

25. A method of validating an assay, comprising:
(1) providing a first DNA library of DNA extracted from tumor cells treated with an apoptosis inducer, wherein the tumor cells have one or more mutations at a chromosomal site;
(2) constructing a second DNA library of DNA prepared from a test sample; and
(3) detecting the one or more mutations from the constructed DNA libraries;
wherein detection of the one or more mutations from the first DNA library indicates the assay is validated; wherein no detection of the one or more mutations from the first DNA library indicates the assay is not validated.

26. (canceled)

27. A method for mimicking human plasma with different DNA mutation frequency, comprising: adding the circulating tumor DNA reference sample of claim 11 into artificial plasma.

28. A cancer prediction kit, comprising the circulating tumor DNA reference sample of claim 11.
